(19) United States
(12) Patent Application Publication
(10) Pub. No.: US 2002/0104148 A1
(43) Pub. Date: Aug. 8, 2002

(54) Male personal hygiene system

(76) Inventor: Sidney I. Silverman, New York, NY (US)

Correspondence Address: Artz & Artz, P.C., Suite 250, 28333 Telegraph Road, Southfield, MI, 48034, US

(21) Appl. No.: 09/778,297

(22) Filed: Feb. 7, 2001

(51) Int. Cl. A61F 13/20; A61F 13/15; A41D 13/04

(52) U.S. Cl. 2/48; 604/385.09; 604/372; 604/375; 604/378; 604/385.03; 604/385.101; 604/387

(57) ABSTRACT

A disposable male personal hygiene product for preventing the soiling of outer clothing and undergarments and skin irritation due to urinary flow irregularities. The product consists of a urine bib having an absorbent outer layer and a moisture resistant inner layer. The urine bib is attached to the male penis during urination by placing the male penis through an opening in the bib such that an absorbent size faces outward and a moisture resistant inner layer is closely coupled to the clothing. The urine bib can be sized as a function of the height and girth of the user. The urine bib may also be treated with a medication to prevent the spread of infectious disease after use and disposal.

### TECHNICAL FIELD

The present invention relates generally to a male personal hygiene product and more particularly relates to a disposable male personal hygiene paper product used for in a specialized sanitary application.

### BACKGROUND

In the anatomy of the male human, urine which collects in the bladder is excreted during the act of urination by passage through the urethra tube, past the prostate gland terminating in the external urethral orifice, or meatus. However, a small amount of residual urine may remain in the urethra. In some adult and senior men, this residual urine can be problemsome. This problem is particularly acute for male adults who experience urinary flow irregularities, such as a sense of urgency, excessive frequency of urination, slow and intermittent urine dribbling on the onset of urination, unpredictable multiple streams of urine during the period of major flow of urine, and unpredictable dribbling at the termination of urination. Often, the residual urine can flow unchecked which can soil the underclothing or trousers and cause acute embarrassment and discomfort. Further, this residual moisture may irritate the skin of the scrotum and perineum. To prevent such delayed flow, it may be possible to forcefully shake the penis over a urinal to discharge the residual urine. During this procedure, however, the path of the exiting urine is uncertain and may contact the hands, trousers, facilities, etc. This situation is not only generally unsanitary, but could lead to the spread of contagious diseases. Although various devices and napkin-like products have been proposed in the past to cope with this problem, these devices have been too expensive or impractical in terms of comfort and ease of use.

Accordingly, it is an object of the present invention to provide a male personal hygiene system that overcomes these difficulties.

### SUMMARY OF THE INVENTION

The above and other beneficial objects and advantages are accomplished in accordance with the present invention by a urine bib (also referred to as a urine shield or urine guard) that used as a protective, moisture-proof, sanitary apron for men who suffer from soiling of trousers and undergarments during periods when they suffer from urinary flow irregularity. The urine bib is adapted from a four-ply paper towel which is plastic backed and consists of multiple layers of absorbent corrugated paper and a layer of moisture-proof, pliable polyethylene, preferably laminated together. It is also possible to crease or fold the bib device in order to assist in directing and absorbing the excessive fluids.

Other objects and advantages of the present invention will become apparent upon considering the following detailed description and appended claims, and upon reference to the accompanying drawings.

### BRIEF DESCRIPTION OF THE DRAWINGS

FIG. 1 is a front plan view of a urine bib according to one embodiment of the present invention;

FIG. 2 is a partial side view of FIG. 1 showing the layers of uncorrugated paper and polyethylene;

FIG. 3 is a front partial view of a urine bib according to another preferred embodiment of the present invention; and

FIG. 4 depicts the use of the urine bib of FIG. 1.

### DESCRIPTION OF THE PREFERRED EMBODIMENT (S)

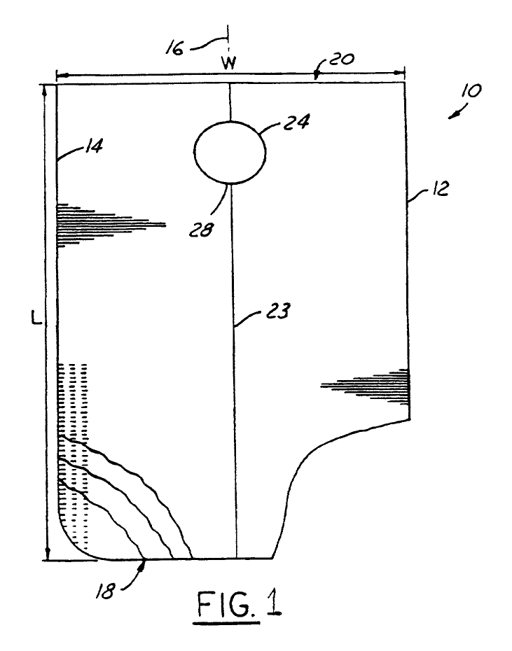

FIG. 1 illustrates a disposable urine bib 10 according to one embodiment of the present invention. The bib 10 comprises a bib body having longitudinal extending side edges 12, 14, a longitudinal length “l,” a longitudinal center line 16, a laterally extending bottom edge 18, a laterally extending top edge 20 and a lateral width “w”. The term “longitudinal” refers to an axis or direction measured along the length “1” of the bib 10 body, which direction or axis is generally parallel to a line extending from the wearer's torso below the wearer's waist as the bib is worn. The terms “lateral” and “transverse” refer to an axis or direction which is perpendicular to the longitudinal center line 16, and which is generally parallel to the line extending across the wearer's waist as the bib 10 is worn. A crease 23 extends along centerline 16 and serves to promote a “v” contour to aid in preventing urine from contacting clothing or undergarments. In the preferred embodiment as depicted in FIG. 1, the opening 24 is about one and one-half inches in diameter and is located approximately one inch below the top edge 20 of the bib 10.

Depending upon the height of the men using the bib 10, the bibs 10 have a variety of different lengths “1”. For example, for a shorter man of approximately five feet tall, the bibs 10 are ten inches long. For taller men, approaching six to seven feet in height, the bibs 10 are fifteen inches long.

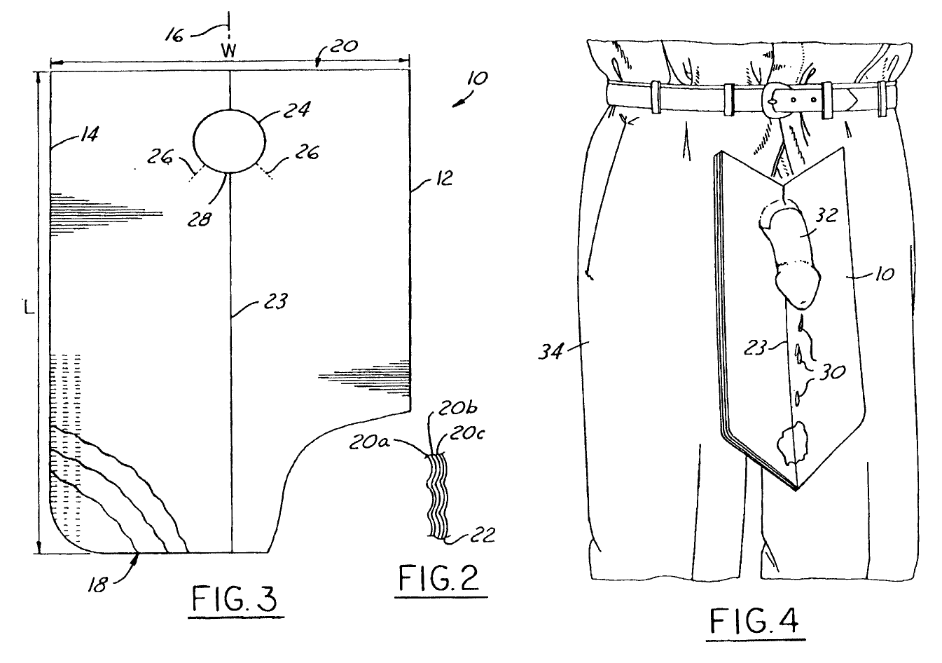

As seen in FIG. 2, the urine bib 10 consists of three layers of absorbent corrugated paper 20a, 20b, 20c and one layer of moisture-proof, but pliable polyethylene 22, all laminated together in techniques that are well known in the art. Each layer of corrugated paper 20a, 20b, 20c and pliable polyethylene 22 are each approximately 0.125 mm thick.

In an alternative embodiment, as depicted in FIG. 3, a series of perforations 26 are closely coupled with the opening 24. The perforations 26 allow the size of the opening 24 to incrementally be enlarged to accommodate larger girth penises comfortably. The number and location of the perforations 26 may vary and still fall within the spirit of the present invention.

As seen in FIG. 4, when the bib 10 is used, a penis 32 is placed through the opening 24 with the polyethylene layer 22 towards the trousers 34. Firmly extending the penis 32 through the opening 24, the bib 10 is elevated to embrace the inferior border (shown as 28 in FIGS. 1 and 3) of the laminated opening around the under surface of the penis. The full extent of the bib 10 is held in a v-contour facing towards the urine receptacle. This v-contour is accomplished by crease 23. The absorbent layers of corrugated paper 20a, 20b, 20c will catch and hold the droplets 30 of urine that would normally fall onto the trousers. After urination is complete, the user is may then use the urine bib 10 to dry any residual urine retained on the penis 32. Next, the soiled urine bib 10 is pulled forward off the penis 32 and disposed either in a hand tower receptacle or in the toilet bowl used for urination.

The present invention offers many advantages not found in prior art in continence appliances. First, the urine bib 10 is easily dispensed and deployed from a storage state. Second, the urine bib 10 is easily disposable within a bathroom setting. Third, this invention is equally useful whether there is a major or minor continence problem, by means of the multiple corrugated absorbent layers of the bib, sufficiently absorbent to contain a substantial amount of liquid. Fourth, as the invention is both compact and light in weight, the invention is thereby made useful of all ages who have a loss of bladder control, such as the young man living with paralysis, the older man with the problem following a prostate surgery, medical facility resident suffering from Alzheimer's Disease or other dementia, and the male of any age with a handicapped condition where continence is a problem. Fifth, the present invention prevents the soiling of one's clothing with the accompanying high cost of laundering and dry-cleaning of trousers 32 and underwear. Sixth, the urine bib 10 is easily sized to accommodate men of different heights by providing different lengths urine bibs. Seventh, the urine bib 10 may be sized to accommodate large girth by incorporating a series of perforating 26. Eighth, the urine bib 10 helps to prevent skin irritation, especially to the scrotum or perineum, that may occur as a result of residual moisture contacting the skin. Ninth, and perhaps most significantly, the present invention helps to relieve the self-deprecating and embarrassing consequences of one's social and behavioral effect in the male who experiences these urinary irregularities.

While the invention has been described in terms of a preferred embodiment, it will be understood, of course, that the invention is not limited thereto since modifications may be made by those skilled in the art, particularly in light of the foregoing teachings. For example, medication, such as an antibiotic, may be added to outer layer of corrugated paper 20a to ensure that selective infectious diseases will not be transmitted or otherwise spread from the bib 10 after use and disposal.

What is claimed is:

1. A disposable urine bib comprising:
at least one outer layer of an absorbent material, said at least one outer layer having a first length and a first width, wherein said at least one outer layer of said absorbent material is capable of absorbing a first quantity of liquid;
a coextensive inner layer of moisture resistant material coupled to an innermost layer of said at least one layer of absorbent material; and
an opening extending through said at least one outer layer and said coextensive inner layer, said opening located approximately equidistant from each side edge of the disposable urine bib and at a position nearer to a top edge of the disposable urine bib, said opening having a first diameter.

2. The disposable urine bib of claim 1, wherein said at least one outer layer of an absorbent material comprises an absorbent corrugated paper.

3. The disposable urine bib of claim 1, wherein said coextensive inner layer comprises a polyethylene layer.

4. The disposable urine bib of claim 1, wherein said at least one outer layer of said absorbent material comprises at least one layer of an absorbent corrugated paper and said coextensive inner layer comprises a polyethylene layer.

5. The urine bib of claim 4, wherein said polyethylene layer and said at least one layer of an absorbent corrugated paper are laminated together to form the urine bib.

6. The urine bib of claim 1, wherein said first length of the urine bib is varied as a function of the height of the user of the urine bib.

7. The urine bib of claim 6, wherein said first length is approximately between ten and fifteen inches.

8. The urine bib of claim 1, wherein said first diameter is approximately one and one-half inches.

9. The urine bib of claim 1 further comprising a series of perforations closely coupled with said opening wherein said series of perforations extend through each of said at least one outer layer and said coextensive inner layer.

10. The urine bib of claim 1, wherein said urine bib has at least one crease extending longitudinally along the length of said urine bib.

11. The urine bib of claim 1, wherein said at least one layer of of an absorbent corrugated paper is treated with a medication, wherein said medication prevents the spread of infectious disease contained within the urine of a male user.

12. A method for preventing soiling of clothing and skin irritation due to urinary flow irregularity in males, the method comprising the steps of:
attaching a urine bib having at least one longitudinally extending crease to a male penis prior to urination, said urine bib having an absorbent outer side capable of absorbing a quantity of urine expelled from the penis during urination and a moisture resistant inner side for preventing said quantity of urine from being desorbed onto the clothing wherein said inner side is in close proximity with the clothing of the male; and
disposing of said urine bib.

13. The method of claim 12, wherein the step of attaching a urine bib comprises the step of:
inserting a male penis through an opening in a urine bib having at least one longitudinally extending crease prior to urination such that an underside of said male penis is embraced against an inferior border of said opening, said urine bib having an absorbent outer side capable of absorbing a quantity of urine expelled from the penis during urination and a moisture resistant inner side for preventing said quantity of urine from being desorbed onto the clothing.

14. The method of claim 12 further comprising the step of wiping said male penis with a dry portion of said absorbent outer side after urination.

15. The method of claim 12 further comprising the step of selecting a length of a urine bib as a function of the height of an intended male user.

16. The method of claim 13, wherein said length of said urine bib is between ten and fifteen inches.

17. A method for preventing soiling of clothing and skin irritation due to urinary flow irregularities, the method comprising the steps of:
providing a urine bib having an opening, said urine bib having an absorbent outer side and a moisture resistant inner side;
sizing said urine bib to accommodate a user penis; inserting said user penis through said opening such that said moisture resistant inner side is closely coupled with the clothing; and
disposing of said urine bib after urination.

18. The method of claim 17, wherein the step of sizing said urine bib comprises the step of selecting a urine bib having a first length, said first length being a function of the height of said user.

19. The method of claim 17 further comprising the step of increasing the size of said opening as a function of the girth of said user penis by tearing at least one of a series of perforations located near said opening.

20. The method of claim 18 further comprising the step of increasing the size of said opening as a function of the girth of said user penis by tearing at least one of a series of perforations located near said opening.
